(19) United States
(12) Patent Application Publication
(10) Pub. No.: US 2024/0164704 A1
(43) Pub. Date: May 23, 2024

(54) Wound Cover to Detect and Monitor a Wound Status, and Method of Using Same

(71) Applicant: Burnam Biotech, LLC, Sammamish, WA (US)

(72) Inventors: Bradley Burnam, Alpharetta, GA (US); Michael H. Burnam, Sammamish, WA (US)

(21) Appl. No.: 18/513,568

(22) Filed: Nov. 19, 2023

Related U.S. Application Data
(60) Provisional application No. 63/384,428, filed on Nov. 20, 2022.

(51) Int. Cl. A61B 5/00; A61B 5/01; A61B 5/026; A61B 5/0531

(52) U.S. Cl. CPC A61B 5/445; A61B 5/0002; A61B 5/01; A61B 5/026; A61B 5/0531; A61B 5/6833; A61B 2560/0406; A61B 2560/045; A61B 2560/0462; A61B 2562/0271

(57) ABSTRACT

The present inventions, in one aspect, are directed to a wound cover to, in operation, be disposed on or over a wound of a user and temporarily affixed to a wound site, having the wound therein, of the user, the wound cover comprising a wound cover stack having a plurality of layers formed in an integrated, unitary structure including a mesh pad layer, a sensor layer, disposed on the mesh pad layer, the sensor layer having a plurality of physiological sensors, configured to detect physiological characteristics and, in response, generate physiological data which are representative thereof, a communication layer configured to output (wired or wirelessly) the data measured or detected by the physiological sensors to circuitry external to the wound cover, and an outer/adhesive layer to, in operation, temporarily affix the wound cover to the wound site of the user.

### RELATED APPLICATION

This non-provisional application claims priority to and the benefit of U.S. Provisional Application No. 63/384,428, entitled “Method and Apparatus to convert a wound cover (bandage) into an information platform for the detection and monitoring of wound status”, filed Nov. 20, 2022. The '428 provisional application is hereby incorporated herein by reference in its entirety.

### INTRODUCTION

There are many inventions described and illustrated herein. These inventions are neither limited to any single aspect nor embodiment thereof, nor to any combination(s) and/or permutation(s) of such aspects and/or embodiments. Importantly, each of the aspects of the present inventions, and/or embodiments thereof, may be employed alone or in combination with one or more of the other aspects of the present inventions and/or embodiments thereof. All combinations and permutations thereof are intended to fall within the scope of the present inventions.

In one aspect, the present inventions are directed to a cover, overlay and/or bandage for a wound, damage or an injury to the body of an animal (e.g., human), for example, a laceration or break in the skin or tissue (whether external or internal), which may be applied and/or temporarily affixed to the body to, for example, protect surface wounds from mechanical trauma and access by infectious agents. The cover, overlay and/or bandage (hereinafter collectively “wound cover” and plural forms thereof) of the present inventions includes an integrated and/or a unitary structure having a plurality of layers wherein at least one of the layers includes one or more sensors capable of measuring, sensing and/or detecting physiological or biological data representative of a current state (and/or change in state) of the wound, damage or an injury (hereinafter, collectively “wound” and plural forms thereof) to the body of an animal. For example, in one embodiment, the wound cover includes an initial layer of porous mesh (e.g., sterile porous mesh) having an exposed, bottom surface that is to be placed on, over and/or in contact with the wound and/or wound site, a second layer (e.g., disposed over and directly contacting the initial layer) having one or more sensors, a third layer (e.g., disposed over and directly contacting the second layer) including electrical, optical and/or inductive communication component(s) (which may also include conductor, cabling or connection elements), and an outer layer (in this exemplary embodiment, a fourth layer which is disposed over and directly contacting the third layer) having an outer adhesive edge or border to adhere the integrated, associated, and/or unitary structure of the wound cover to the skin of the animal beyond the wound site.

In one embodiment, an electrical conductor or cable, which is connected to the communication component(s) of the third layer and/or the sensor(s) of the second layer, may be affixed to or provided through the outer/adhesive layer to facilitate transmission of the sensor-derived biological data to circuitry external to the wound cover (e.g., local and/or remote circuitry (e.g., signal acquisition and processing circuitry)). Notably, additional layers may be included between the aforementioned initial/first, second, third and/or fourth/outer layers of the aforementioned exemplary wound cover.

The one or more sensors of the wound cover, in one embodiment, may sense or detect biological data of (or representative of), for example, the wound and/or the wound site of the animal (e.g., human) including, for example, one or more sensors to measure a temperature (e.g., temperature of the (i) tissue of the wound, (ii) skin adjacent to wound and/or (iii) surrounding the wound), chemical sensor (e.g., pH or water consistency/quality in the wound, adjacent to the wound and/or surrounding the wound), bioimpedance (e.g., local bioimpedance for tissue fluid monitoring of the region adjacent to, surrounding and/or including the wound), blood flow (e.g., local blood flow in and/or adjacent to the wound and/or in a region including the wound site (e.g., a local region surrounding or adjacent to the wound)—photoplethysmography as a measure thereof), autonomic nervous system (ANS) tone, and/or skin condition (e.g., color of the (i) tissue of the wound, (ii) skin adjacent to the wound, and/or (iii) surrounding the wound), and/or change(s) in temperature, chemistry (e.g., pH), bioimpedance, blood flow, ANS tone, and/or skin condition of, for example, the wound and/or the wound site of the animal.

Thus, in one embodiment, physiologic status and/or physiologic changes in the state of the wound and/or wound site monitored by the wound cover include one or more of the following: (i) increased temperature, (ii) increased redness, (iii) chemical changes associated with purulent drainage, (iv) swelling, or local tissue accumulation, and/or (v) excess local sympathetic tone. Importantly any sensor(s), and/or physiologic status or changes in the state of the wound and/or wound site, whether now known or later developed, are intended to fall within the scope of the present inventions.

The one or more sensors may intermittently, periodically and/or continuously sense or detect biological data. In this way, the wound cover of the present inventions may provide intermittently, periodically and/or continuously biological data which is/are representative of a current state (and/or change in state) of the wound, damage or an injury to the body of an animal—which may be tantamount to suitable inspection of the wound, damage or an injury associated with the wound cover—without removal of the cover, overlay and/or bandage and the inherent exposure of the covered site to, for example, the local environment, which may be disruptive to the healing process and risk outside contamination of the wound, damage or an injury associated with the wound cover. Indeed, the wound cover of the present inventions may provide biological data which is/are representative of a current state (and/or change in state) of the wound, damage or an injury to the body of an animal under those circumstances where suitable inspection of a covered site associated with the wound is problematic, costly and/or time consuming—for example, where the wound, damage and/or an injury is located beneath or is covered by a “fixed” solid cast (e.g., a conventional plaster cast providing a hard covering that protects, stabilizes and/or immobilizes a limb and/or joint).

The sensor(s) may include one or more active-type sensor(s) that are powered (e.g., via a local and/or remote electrical power sources—such as, for example, a thermocouple and bioimpedance sensor) and/or one or more passive-type sensor(s) that are not powered (e.g., a thermistor or temperature sensitive strip) wherein each passive-type sensor detects, senses and/or measures a biological condition of the wound and/or wound site, and/or responds to an output therefrom, for example, from the wound (e.g., a sensor that generate an output signal(s) without application of electrical power) such as a sensor that detects temperature (e.g., heat) or a change in temperature. Moreover, in one embodiment, the sensor(s) may be disposed in one layer of the plurality of layers of the integrated and/or a unitary structure of the wound cover. In this embodiment, the wound cover includes one layer having one or more sensors. In another embodiment, one or more sensor(s) may be disposed in each of a plurality of layers of the wound cover and, as such, in this embodiment the wound cover includes sensors in a plurality of layers, each layer including one or more sensors.

In one embodiment, the wound cover includes one or more electrical power sources resident therein/thereon (e.g., a battery) which is configured to power circuitry of the wound cover and/or one or more sensors to, among other things, intermittently, periodically and/or continuously sense or detect biological data. In addition thereto, or in lieu thereof, in another embodiment, the wound cover is configured to receive electrical power from an external source via one or more electrical conductors or cables, which may after be distributed to the sensor(s) and/or other circuitry of the wound cover (e.g., communication component(s)).

The wound cover, in another embodiment, may also include communication circuitry to facilitate communication/transmission of the biological data detected by one or more of the active or passive sensors to circuitry that is external to the wound cover. Here, the communication circuitry may transmit the data to, for example, one or more local and/or wearable electronic devices, remote circuitry, and/or remote device(s) or facility(ies). Notably, the communication/transmission may be wired and/or wireless, and may be based on, for example, optical, inductive, capacitive and/or electronic techniques. All forms of communication/transmission are intended to fall within the scope of the present inventions.

Notably, one or more (or all) of the layers may include a plurality of components and/or perform a plurality of functions/operations/purposes—for example, the initial layer that is intended to contact and/or directly cover the wound of the wound site (e.g., mesh pad/layer) may, in addition, include one or more sensors to detect, sense and/to measure biological data representative of a current state (and/or change in state) of the wound or wound site. In that case, the mesh pad/layer is also a sensor layer. In addition, the sensor layer may also include features/components of a connector/cabling layer, a communication layer, and/or a circuitry layer. Again, in that case, the sensor layer is also connector/cabling layer, a communication layer and/or a circuitry layer, as the case may be. Thus, each layer of the wound stack of the sound cover is not limited (unless expressly stated otherwise) to a single function, operation, purpose, and/or component type. (e.g., connector/cabling, sensor, communication, circuitry (e.g., processing).

In addition thereto, or in lieu thereof, a wound cover reader may be employed to optically, inductively and/or electronically detect and/or read the biological data from the sensor(s) of the wound cover. Here, a wound cover reader (for example, a handheld device) may acquire, detect and/or read the biological data from the sensor(s), via, for example, positioning the wound cover reader in operable proximity (optically, inductively and/or electronically) to the wound cover (e.g., the sensor(s) and/or communication circuitry connected to the sensor(s)) and, in that way, acquire the physiological data which are representative of a current state (and/or change in state) of the wound, damage or an injury to the body of an animal. In one embodiment, the sensor(s) may be passive-type sensor(s) wherein the wound cover reader, via wireless transmission of electrical energy and/or near-field communication (NFC) techniques/circuitry may (i) energize, interrogate, enable, and/or access one or more sensors which, in response, may measure, collect and/or detect associated data and, in addition thereto, or in lieu thereof, (ii) transmit and/or output such data (via a transmitter) to the wound cover reader. As such, in this embodiment, the wound cover reader may wirelessly power the sensor(s) of the wound cover and/or directly acquire the physiological data in and/or from such sensor(s)

Accordingly, the wound cover reader may read (e.g., optically, inductively and/or electronically) the data directly from the sensor(s) and/or via communication circuitry of the wound cover—for example, (i) optically reads the temperature detected by a temperature sensitive strip (e.g., the color of the strip) and/or (ii) inductively and/or electronically read the temperature detected by a thermocouple or thermistor (e.g., the voltage, current and/or resistance output of a thermocouple or thermistor, which is in operable proximity of the wound, via wireless transmission of electrical energy and/or NFC techniques/circuitry). Notably, “NFC techniques/circuitry” means techniques and/or circuitry to implement wireless transmission of (a) electrical energy and/or (b) data and/or control, including techniques that and/or circuitry configured to: (i) energize, interrogate, enable, and/or access one or more sensors which, in response, may measure, collect and/or detect associated data, and/or (ii) transmit and/or output such data (via a transmitter in the wound cover, e.g., a transmitter in a sensor in the wound cover) to a receiver in the wound cover reader.

As such, in addition to transmission of data wirelessly from one or more sensors of the wound cover to the wound cover reader, or in lieu thereof, the wound cover reader may, in one embodiment, provide one or more sensors of the wound cover power/energy, via NFC techniques/circuitry, to measure, collect and/or detect associated physiological. Thus, in one embodiment, circuitry in the wound cover reader, when in an operable proximity of the sensor(s), provides and/or supplies power/energy to such sensor(s) (e.g., inductively and/or electronically) to sense, measure and/or detect physiological data corresponding to the sensor, and thereafter transmit and/or output such data to the wound cover reader via NFC techniques/circuitry. Here, the wound cover reader is providing or supplying the physiological sensor(s) power/energy to both sense/detect data and transmit/output that data to, for example, the wound cover reader.

An exemplary wound cover implementing such a configuration may include an initial layer of porous mesh (e.g., sterile porous mesh) in contact with the wound, a second layer (e.g., disposed over and directly contacting the initial layer) having one or more sensor(s)—for example, one or more thermocouples and/or thermistors, and/or one or more temperature sensitive strips, and an outer, top, exposed layer (in this exemplary embodiment, a fourth layer which is disposed over and directly contacting the third layer) having an adhesive edge or border to adhere the integrated, associated, and/or unitary structure of the wound cover to the skin of the animal beyond the wound site. In this embodiment, a communication component layer (e.g., disposed over and directly contacting the sensor layer) including electrical, optical and/or inductive communication component(s) may be omitted wherein the wound cover reader may be employed to facilitate acquisition of the sensor-derived biological data and, in one embodiment, may further be employed to transmit such data to circuitry external to the wound cover (e.g., local and/or remote circuitry (e.g., a signal acquisition and processing module)). Notably, again additional layers may be included between the initial/first, second, third/outer layers of this exemplary wound cover.

In one embodiment, the wound cover reader (e.g., the handheld device) may display state/status and/or change in state/status of wound, damage or an injury to the body of an animal. In one embodiment, biological data representative of a current state (or change in state) of the wound, damage or an injury may thereafter be provided (for example, in real-time, e.g., via the Internet or an intranet) to a medical professional (whether local or remote) for evaluation or consideration. Notably, in addition to input from a medical professional, or in lieu thereof, the wound cover and system of the present inventions may employ artificial intelligence (AI) methods/processes for data analysis, trending and/or evaluation. This would be beneficial both to the patient and healthcare economic system as a whole by reducing the need for additional medical services and by returning the patient to productivity sooner. Indeed, in one aspect, the wound cover facilitates monitoring the wound and/or wound site for infection by “sensing” changes in physiological condition of the user, for example, in real-time, continuously, intermittently and/or periodically, with the aid of AI and machine learning methods/processes.

With the aforementioned in mind, the design of conventional covers or bandages often prohibit/inhibit proper visual inspection without removal which may frustrate and/or prevent detection of signs of infection or other cause of failure of the wound to heal. For example, conventional covers or bandages include traditional gauze style wound dressings, telfa pads, alginates, foams, and antimicrobial impregnated versions of all of those mentioned. Conventional covers or bandages often include an adhesive border, while some require paper and/or equivalent medical tape for affixation. Such covers or bandages are intended to remain in place over or on the covered site and undisturbed—thereby inhibiting or prohibiting inspection of the covered site until removal and/or changes of wound dressing—which may be inherently disruptive to the healing process and risk outside contamination of the site. Conventional bandages, although in direct contact with the wound, do not sense local tissue characteristics that would be useful in detecting and monitoring infection, wound trauma, other causes of inflammation, or fluid accumulation.

Briefly, infections are one of the most serious complications associated with wounds. Even for wounds resulting from clean surgery, studies find the infection rate to be 8% among the general population, and 25% among those above 60 years old. Covering a break in the skin caused by trauma or surgery helps to control bleeding and protect against infection. Typical wound dressings are pads of gauze or cloth that can be placed directly against the wound to absorb blood and other fluids. Cloth bandages often cover dressings and hold them in place. In the 8-25% of patients who eventually suffer wound infection despite wound covers, such covers become a negative factor for detecting and monitoring infection by preventing information access effectively blinding the caregiver to available information such as the physical characteristics of the wound, temperature, redness, swelling, even odor. While removing the bandage periodically mitigates this somewhat, it also exposes the wound to further trauma and/or infection, and fails to provide the medical professional the ability to obtain frequent and current wound states “on demand” or in a timely fashion, and without disruption to the healing process and risk outside contamination of the wound site that is accompanied with removal and/or changes of wound dressing.

Initial signs of infection are often visual and tactile, such as redness, streaks, fluid accumulation, swelling, pH changes, odor, and pain at the site. Infections may develop in as short as three to five days. Traditional wound dressing changes may be up to seven days. Depending upon the site of infection, such as in the case of a post-operative wound after implantable hardware surgery, sepsis can be rapid, possibly commencing before the initial dressing change. Changes in the local anatomy associated with a wound may be rapid and conventional wound dressings fail to “alert” the caregiver to their existence and progression.

As noted herein, there are many inventions described and illustrated herein. These inventions are neither limited to any single aspect nor embodiment thereof, nor to any combination(s) and/or permutation(s) of such aspects and/or embodiments. Importantly, each of the aspects of the present inventions, and/or embodiments thereof, may be employed alone or in combination with one or more of the other aspects of the present inventions and/or embodiments thereof. Additional features and/or advantages will be apparent to one skilled in the art from the description, drawings, and/or claims.

### BRIEF DESCRIPTION OF THE DRAWINGS

The present inventions may be implemented in connection with embodiments illustrated in the attached drawings. These drawings show different aspects of the present inventions and, where appropriate, reference numerals illustrating like structures, components, materials and/or elements in different figures are labeled similarly. It is understood that various combinations of the structures, components, and/or elements, other than those specifically shown, are contemplated and are within the scope of the present inventions.

Moreover, there are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

The inventions are not limited to the illustrative/exemplary embodiments set forth in this application. Again, there are many inventions described and illustrated herein. The present inventions are neither limited to any single aspect nor embodiment thereof, nor to any combinations and/or permutations of such aspects and/or embodiments. Each of the aspects of the present inventions, and/or embodiments thereof, may be employed alone or in combination with one or more of the other aspects of the present inventions and/or embodiments thereof. For the sake of brevity, many of those combinations and permutations are neither illustrated nor discussed separately herein.

FIG. 1A illustrates, in a schematic block diagram form, an exemplary embodiment of a wound cover includes a wound cover stack having plurality of layers including (i) at least one of which is a sensor layer and (ii) an outer, top, exposed, adhesive layer which, in operation, is configured or capable of securing the wound cover to the skin of the animal (e.g., human) via an adhesive edge or border, according to one or more embodiments of the present inventions;

FIG. 1B illustrates a cross-sectional view, in schematic block diagram form, of the exemplary embodiment of the wound cover of FIG. 1A along line A:A (see FIG. 1) wherein the wound cover stack, in this illustrative embodiment, includes, from bottom (which is intended to contact and/or directly cover the wound of the wound site) to top, a mesh pad or layer having an exposed, bottom surface that is configured to be placed on, over and/or in contact with the wound and/or wound site (generally illustrated as a dotted box), one or more sensor layers disposed on the mesh pad or layer, a connector/cabling layer to facilitate output of the data measured or detected by the sensors of the senor layer(s) to, in one embodiment, circuitry external to the wound cover, and an outer, top, exposed layer having an adhesive edge or border, which extends beyond the other layers of the wound cover stack, to, in operation, adhere and/or temporarily affix the integrated, unitary structure of the wound cover to the wound and, in this illustrative embodiment, the skin of the animal in the immediate vicinity of the wound (i.e., the wound site), according to one or more embodiments of the present inventions; other techniques, configurations and materials may be employed to adhere and/or temporarily affix the integrated, unitary structure of the wound cover to the wound and/or wound site (e.g., medical tape, bandage wrap, cast and/or lightweight harness) and are intended to fall within the scope of the present inventions;

FIG. 1C illustrates, in schematic block diagram form, an exemplary wound cover (with outer, top layer removed or being transparent so that layers beneath the outer layer are “visible”), according to one or more embodiments of the present inventions, wherein, in this illustrative embodiment, includes, from bottom (which is intended to contact and/or directly cover the wound of the wound site) to top, a mesh pad or layer to contact the wound, one or more sensor layers, and a connector/cabling layer to facilitate output of the data acquired by the sensors of the senor layer(s) to circuitry external to the wound cover; in this illustrative example, the sensor layer includes a plurality of sensor types including chemical sensor(s), an ANS sensor, a PPG sensor, a thermistor and/or thermocouple, and bioimpedance sensor; this sensor layer is merely illustrative and the sensor layer may include more or less sensor types as well as one or more other sensor types—all combinations and permutations of sensor types are intended to fall within the scope of the present inventions; an electrical cable is employed to facilitate communication of the sensor data to a processor module which, in one embodiment, may communicate such data to data storage or processing circuitry in the “cloud”; notably, although the wound is generally illustrated, in practice or implementation, the wound and wound site is likely to be obscured via the layers of the wound cover stack which are placed over the wound and wound site;

FIG. 1D illustrates, in schematic block diagram form, the configuration of the different portions, components or features of a system including the wound cover of FIG. 1C, including a roster or list of the various components (e.g., sensors and circuitry) and features of each aspect of such system;

FIG. 1E illustrates, in schematic block diagram form, an exemplary wound cover (with outer, top layer removed or being transparent so that layers beneath the outer layer are “visible”), according to one or more embodiments of the present inventions, wherein, in this illustrative embodiment, includes, from bottom (which is intended to contact and/or directly cover the wound of the wound site) to top, a mesh pad or layer to contact the wound, one or more sensor layers, and a connector/cabling layer to facilitate output of the data acquired by the sensors of the senor layer(s) to circuitry external to the wound cover; in this illustrative example, the sensor layer includes a plurality of sensor types including chemical sensor(s), an ANS sensor, a PPG sensor, a thermistor and/or thermocouple, and bioimpedance sensor; again this sensor layer is merely illustrative and the sensor layer may include more or less sensor types as well as one or more other sensor types—all combinations and permutations of sensor types are intended to fall within the scope of the present inventions; in this embodiment, the wound cover implements wireless communication to facilitate transfer of the sensor data to a processor module which, in one embodiment, may communicate such data to data storage or processing circuitry in the “cloud”; notably, although the wound is generally illustrated, in practice or implementation, the wound and wound site is likely to be obscured via the layers of the wound cover stack which are placed over the wound and wound site;

FIG. 1F illustrates, in schematic block diagram form, the configuration of the different portions, components or features of a system including the wound cover of FIG. 1E, including a roster or list of the various components (e.g., sensors and circuitry) and features of each aspect of such system;

FIG. 1G illustrates, in schematic block diagram form, an exemplary wound cover (with outer, top layer removed or being transparent so that layers beneath the outer layer are “visible”), according to one or more embodiments of the present inventions, wherein, in this illustrative embodiment, includes, from bottom (which is intended to contact and/or directly cover the wound of the wound site) to top, a mesh pad or layer to contact the wound, one or more sensor layers, and a connector/cabling layer to facilitate output of the data acquired by the sensors of the senor layer(s) to circuitry external to the wound cover; in this illustrative example, the sensor layer includes a plurality of sensor types including chemical sensor(s), an ANS sensor, a PPG sensor, a thermistor and/or thermocouple, and bioimpedance sensor; again this sensor layer is merely illustrative and the sensor layer may include more or less sensor types as well as one or more other sensor types—all combinations and permutations of sensor types are intended to fall within the scope of the present inventions; in this embodiment, the wound cover implements wireless communication to facilitate transfer of the sensor data to a processor module which, in one embodiment, may perform, execute and/or assess one or more of the functions or operations of assessing the sensor data; notably, in each and every embodiment, the wound cover may including one or more data storage elements;

FIG. 1H illustrates, in schematic block diagram form, the configuration of the different portions, components or features of a system including the wound cover of FIG. 1G, including a roster or list of the various components (e.g., sensors and circuitry) and features of each aspect of such system;

FIGS. 2A to 2G illustrate, in schematic block diagram form, exemplary embodiments of a plurality of layers of a wound stack of a wound cover according to one or more embodiments of the present inventions, wherein the wound stack is depicted from a bottom of mesh pad layer, which is intended to contact and/or directly cover the wound of the wound site, to a top of the outer layer, which is configured, in one embodiment to include an adhesive edge or border which is configured to adhere or capable of temporarily affixing the integrated, associated, and/or unitary structure of the wound cover to the skin of the animal (including the wound) in and/or around a wound site;

FIG. 3A illustrates, in schematic block diagram form, the sensor layer(s) may include a plurality of layers such that one or more sensor(s) may be disposed in each of a plurality of layers of the wound cover according to one or more embodiments of the present inventions, wherein in this illustrative embodiment, the sensor layers of the wound cover include one or more sensors in each of the plurality of layers;

FIG. 3B illustrates, in block diagram form, exemplary physiological sensor(s) to determine, sense, detect, assess and/or obtain data/information which are representative of physiological condition of the wound and/or wound site of the user, according to at least certain embodiments of the present inventions;

FIG. 4 illustrates, in block diagram form, an exemplary embodiment of a system including a wound cover, of the present inventions, and a wound cover reader, according to one or more embodiments of the present inventions, wherein the wound cover reader is configured to, for example, detect and/or read (implementing wired and/or wireless communication) the biological or physiological condition(s) of the wound and/or wound site of the user that is/are detected, measured and/or sensed by one or more of the sensors of the sensor layer(s) of the wound cover, according to one or more embodiments of the present inventions; in one embodiment, the wound cover reader employs NFC techniques/circuitry to wirelessly obtain and/or acquire the biological or physiological data detected, measured and/or sensed by one or more of the sensors of the sensor layer(s) of the wound cover, according to one or more embodiments of the present inventions; notably, in addition thereto, or in lieu thereof, the wound cover reader may provide the sensor(s) power/energy to enable, measure, collect and/or detect associated physiological (e.g., inductively and/or electronically)—also via NFC techniques/circuitry;

FIGS. 5A-5C illustrate, in block diagram form, exemplary embodiments of wound cover readers (for example, handheld devices) to obtain, acquire and/or read the biological or physiological condition(s) of the wound and/or wound site of the user detected, measured and/or sensed by one or more of the sensors of the sensor layer(s) of the wound cover, according to one or more embodiments of the present inventions; the wound cover reader includes a sensor reader (e.g., NFC techniques/circuitry), which may, among other things, optically, inductively and/or electronically obtain, acquire and/or read the data generated, detected and/or measured by the sensor(s) of the wound cover, according to one or more embodiments of the present inventions; in one embodiment, the sensor reader implements NFC techniques/circuitry to acquire the biological or physiological data wirelessly including employing NFC techniques/circuitry to provide the sensor(s) power/energy to enable, measure, collect and/or detect associated physiological (e.g., inductively and/or electronically) and thereafter optically, inductively and/or electronically access the sensors to acquire or read such data; in one embodiment, the wound cover reader may be a handheld and portable device which includes communication circuitry (e.g., a wireless transmitter) to: (i) output, transmit and/or provide data which are representative of the biological or physiological condition(s) of the wound and/or wound site of the user as detected or measured by the sensor(s); the handheld portable wound cover reader may include a display to visually output information (e.g., condition(s) of the wound and/or wound site) and a user interface having an input mechanism (for example, a touch screen overlay for a display and/or buttons, wherein the user may input data and/or commands) and/or processor circuitry to evaluate, process, filter and/or analyze the physiological data which are representative of a current state (or change in state) of the wound wherein a medical professional (e.g., local professional operating the wound cover reader) may evaluate such data and provide recommendations of a course of action; in certain embodiments the wound cover reader also includes certain communication circuitry to provide (for example, in real-time, e.g., via the Internet or an intranet) the sensor data of the wound cover to a medical professional (whether local or remote) for evaluation or consideration;

FIG. 6 illustrates, in block diagram form, an exemplary system embodiment of a wound cover reader, which is configured to (i) connect, via wired and/or wireless techniques, directly to a wound cover (see, e.g., FIGS. 1A-1H and 2A-2G) and (ii) connect, via wired and/or wireless techniques, directly to remote circuitry (e.g., a server and/or storage) in the Internet or remote processing system (e.g., one or more servers in the “cloud” or on the “edge” of the “cloud”—over the air (OTA)), according to one or more embodiments of the present inventions; in one embodiment, the Internet or remote processing system may transmit data/control corresponding to corrective intervention and/or appropriate treatment to the wound cover reader wherein, in response, the reader may provide (e.g., visually and/or audibly) corrective intervention and/or appropriate treatment to a medical professional to implement corrective intervention and/or appropriate treatment;

FIG. 7A illustrates, in block diagram form, an exemplary embodiment of a system including a wound cover, of the present inventions, and a portable electronic device, according to one or more embodiments of the present inventions, wherein the portable electronic device (e.g., mobile phone or smartwatch) is configured to, for example, acquire and/or read (implementing wired and/or wireless communication) the biological or physiological condition(s) of the wound and/or wound site of the user that is/are detected, measured and/or sensed by one or more of the sensors of the sensor layer(s) of the wound cover, according to one or more embodiments of the present inventions; and

FIG. 7B illustrates, in block diagram form, an exemplary system embodiment of a portable electronic device (e.g., mobile phone or smartwatch), which is configured to (i) connect, via wired and/or wireless techniques, directly to a wound cover (see, e.g., FIGS. 1A-1H and 2A-2G) and (ii) connect, via wired and/or wireless techniques, directly to remote circuitry (e.g., a server and/or storage) in the Internet or remote processing system (e.g., one or more servers in the “cloud” or on the “edge” of the “cloud”—over the air (OTA)), according to one or more embodiments of the present inventions; in one embodiment, the Internet or remote processing system may transmit data/control corresponding to corrective intervention and/or appropriate treatment to the portable electronic device wherein, in response, the device may provide (e.g., visually and/or audibly) corrective intervention and/or appropriate treatment to a medical professional to implement corrective intervention and/or appropriate treatment.

Again, there are many inventions described and illustrated herein. The present inventions are neither limited to any single aspect nor embodiment thereof, nor to any combinations and/or permutations of such aspects and/or embodiments. Each of the aspects of the present inventions, and/or embodiments thereof, may be employed alone or in combination with one or more of the other aspects of the present inventions and/or embodiments thereof. For the sake of brevity, many of those combinations and permutations are not discussed or illustrated separately herein.

### DETAILED DESCRIPTION

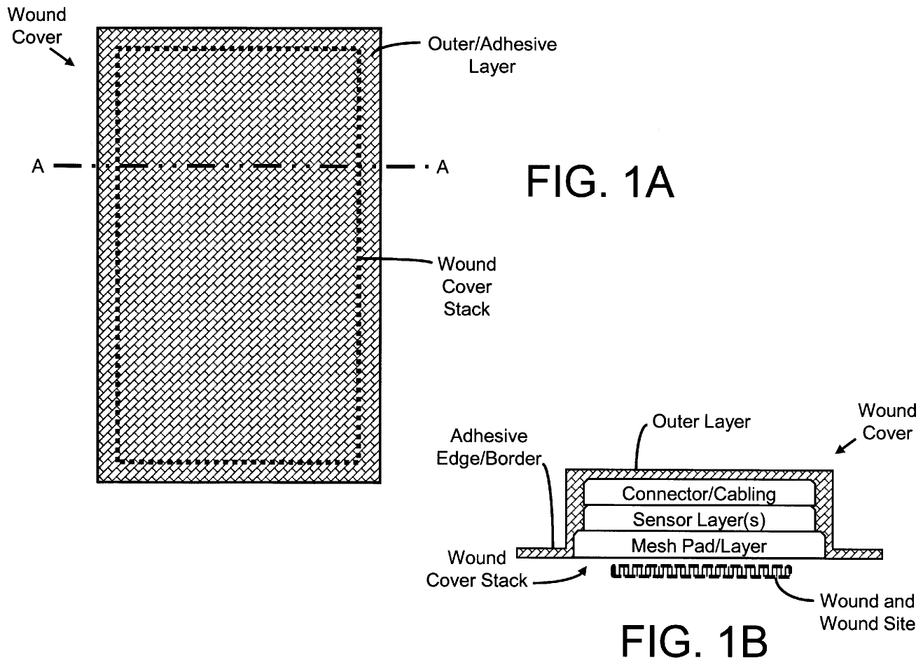

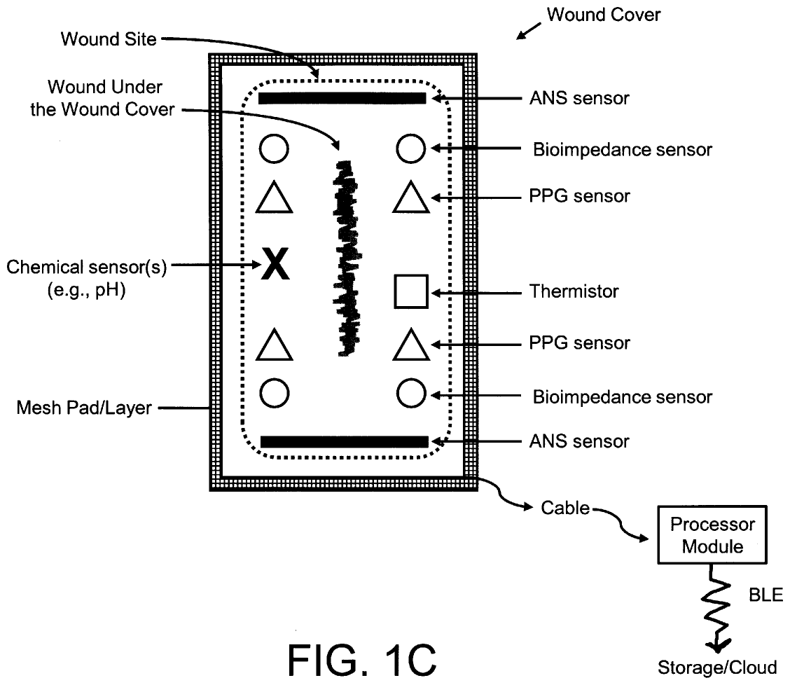

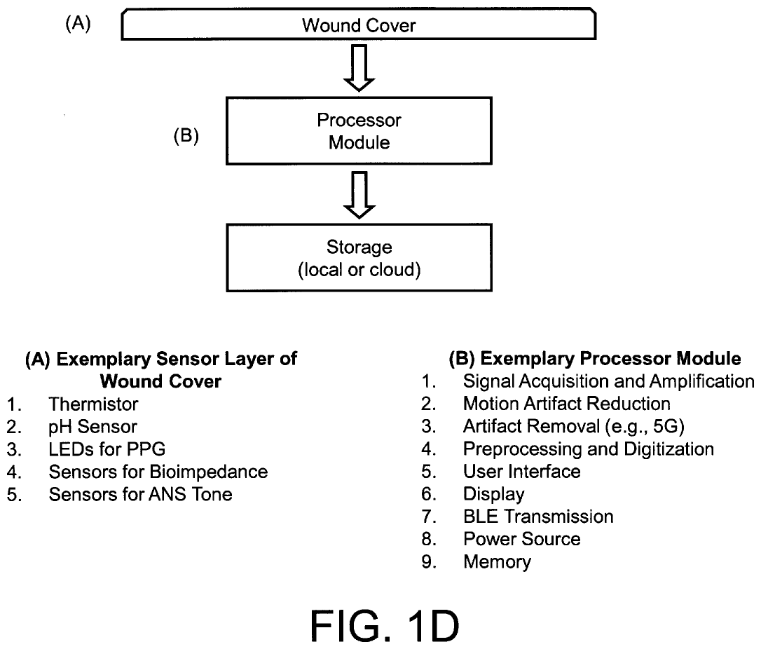

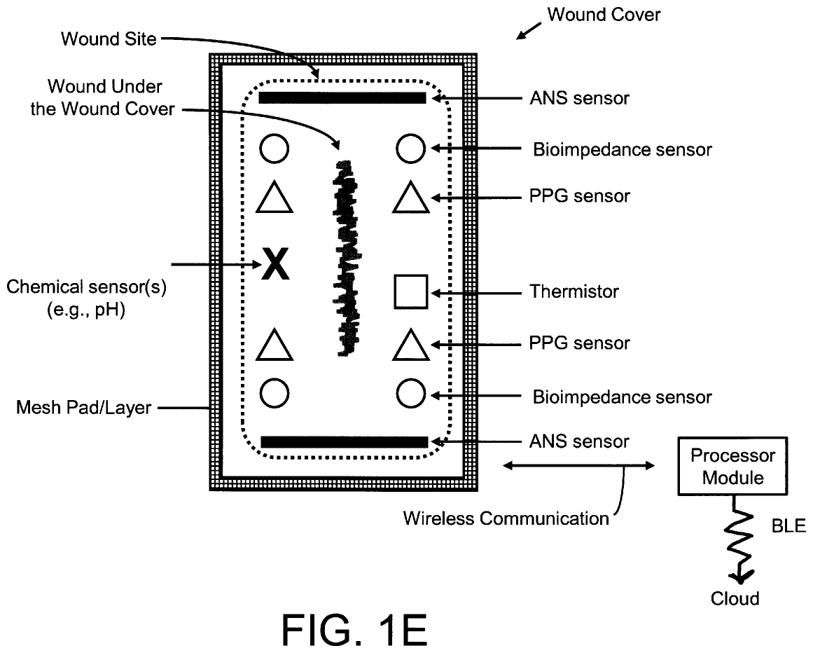

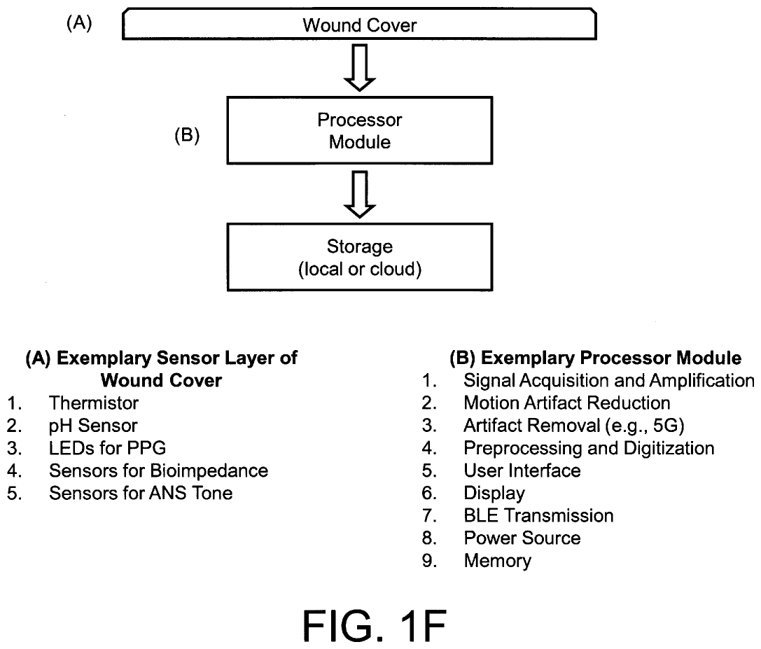

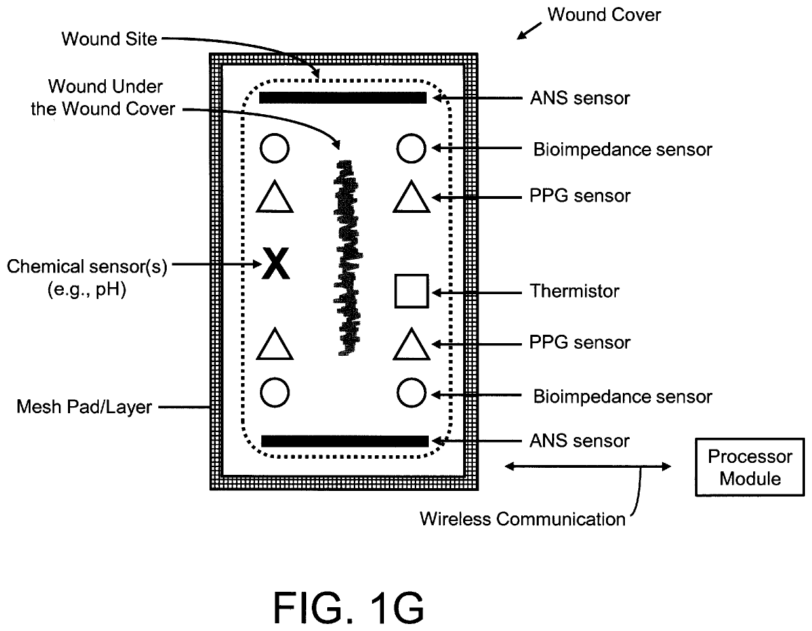

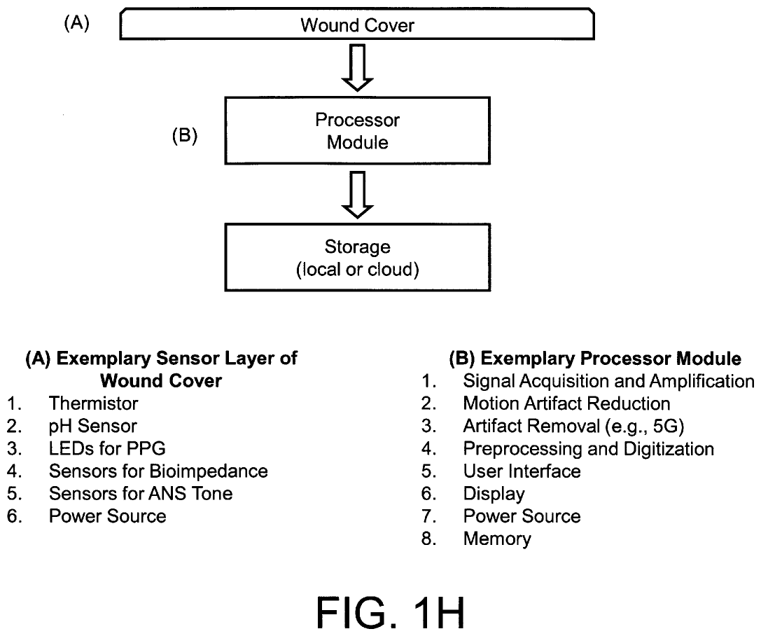

In one aspect, the present inventions are directed to a wound cover to overlay on a wound and/or wound site of a body of an animal (e.g., human), for example, a laceration or break in the skin or tissue (whether external or internal) to, for example, protect surface wounds from mechanical trauma and access by infectious agents. The wound cover of the present inventions includes an integrated and/or a unitary structure having a plurality of associated layers wherein at least one of the layers includes one or more sensors capable of measuring, sensing and/or detecting physiological or biological data representative of a current state (and/or change in state) of the wound and/or wound site of the body of an animal. For example, with reference to FIGS. 1A and 1B, in one embodiment, the wound cover includes an initial layer of porous mesh (e.g., sterile porous mesh pad) having an exposed, bottom surface that is to be placed on, over and/or in contact with the wound and/or wound site (generally illustrated as a dotted box in FIG. 1B), and a second layer (e.g., disposed over and directly contacting the initial layer) having one or more sensors to measure, sense and/or detect physiological or biological data representative of a current state (and/or change in state) of the wound and/or wound site. A third layer (e.g., disposed over and directly contacting the sensor layer) may include electrical, optical and/or inductive communication component(s) (which, in one embodiment, consists of one or more conductors and/or connectors). A final or outer layer (in this exemplary embodiment, a fourth layer which is disposed over and directly contacting the third layer) is configured to contain and/or maintain the wound cover stack in an integrated and/or a unitary structure. Here, the outer, top, exposed layer may include an adhesive material including an adhesive border or edge, which extends beyond the other layers of the wound cover stack, to adhere and/or temporarily affix the integrated, unitary structure of the wound cover to the wound and/or wound site and, in this illustrative embodiment, the skin of the animal in the immediate vicinity of the wound (i.e., the wound site).

With reference to FIGS. 1B and 1C, in one embodiment, an electrical conductor or cable, which is connected to the sensors of the sensor layer and/or the communication component(s) of the third layer, may be affixed to or provided through the outer/adhesive layer to facilitate transmission of the sensor-derived physiological data to circuitry external to the wound cover (e.g., local and/or remote circuitry (e.g., signal acquisition and processing circuitry)). Notably, additional layers may be included between the aforementioned initial/first, second, third and/or fourth/outer layers of the aforementioned exemplary wound cover. In one embodiment, the electrical conductor or cable is connected to a processor module which may include one or more state machines and/or one or more processors to perform data analyses, trending and/or evaluations. The processor module may additional circuitry including circuitry to transmit the sensor data (e.g., raw data) and/or data of the analyses, trending and/or evaluations to other circuitry including data storage. (See, e.g., FIG. 1D).

With reference to FIGS. 1C-1H, the wound cover, in another embodiment, may include and/or facilitate wireless communication circuitry to implement communication or transmission of the biological data detected by one or more of the sensors to circuitry that is external to the wound cover—in these illustrated exemplary embodiment, a processor module. The communication circuitry may transmit the data to, for example, one or more local and/or wearable electronic devices, remote circuitry, and/or remote device(s) or facility(ies). Notably, the communication or transmission of sensor data may be wired and/or wireless, and may be based on, for example, optical, inductive, capacitive and/or electronic techniques. All forms of communication or transmission are intended to fall within the scope of the present inventions.

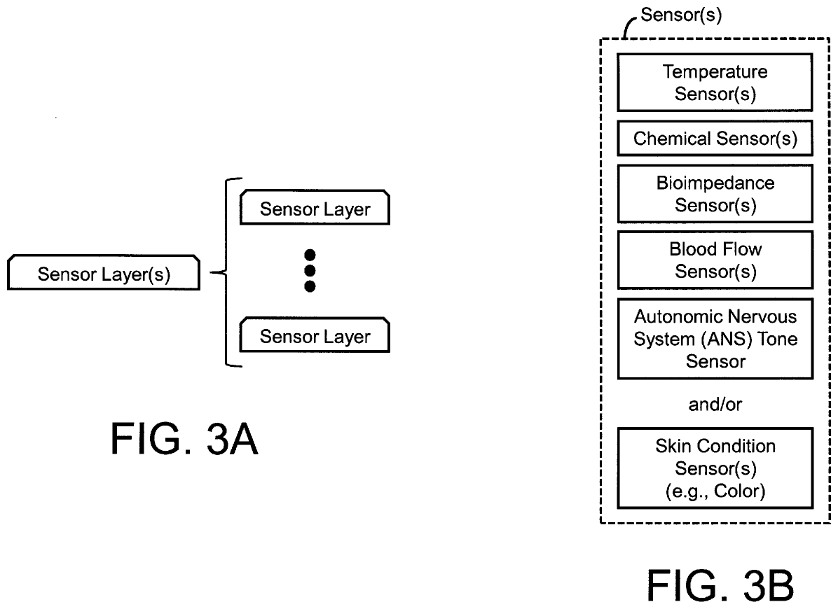

With continued reference to FIGS. 1C-1H and with reference to FIG. 3B, the one or more sensors of the wound cover, in one embodiment, may sense or detect biological data of (or representative of), for example, the wound and/or the wound site of the animal (e.g., human) including, for example, one or more sensors to measure a temperature (e.g., temperature of the (i) tissue of the wound, (ii) skin adjacent to wound and/or (iii) surrounding the wound), chemical sensor (e.g., pH or water consistency/quality in the wound, adjacent to the wound and/or surrounding the wound), bioimpedance (e.g., local bioimpedance for tissue fluid monitoring of the region adjacent to, surrounding and/or including the wound site), blood flow (e.g., local blood flow in and/or adjacent to the wound and/or in a region including the wound site (e.g., a local region surrounding or adjacent to the wound site)—photoplethysmography as a measure thereof), autonomic nervous system (ANS) tone, and/or skin condition (e.g., color of the (i) tissue of the wound, (ii) skin adjacent to the wound, and/or (iii) surrounding the wound), and/or change(s) in temperature, chemistry (e.g., pH), bioimpedance, blood flow, ANS tone, and/or skin condition of, for example, the wound and/or the wound site of the animal.

Thus, in one embodiment, physiologic status and/or physiologic changes in the state of the wound and/or wound site monitored by the wound cover include one or more of the following: (i) increased temperature, (ii) increased redness, (iii) chemical changes associated with purulent drainage, (iv) swelling, or local tissue accumulation, and/or (v) excess local sympathetic tone. Importantly, any sensor(s), and/or physiologic status or changes in the state of the wound and/or wound site, whether now known or later developed, are intended to fall within the scope of the present inventions.

The one or more sensors may intermittently, periodically and/or continuously sense or detect physiological data. In this way, the wound cover of the present inventions may provide intermittently, periodically and/or continuously biological data which is/are representative of a current state (and/or change in state) of the wound, damage or an injury to the body of an animal—which may be tantamount to suitable inspection of the wound, damage or an injury associated with the wound cover—without removal of the cover, overlay and/or bandage and the inherent exposure of the covered site to, for example, the local environment, which may be disruptive to the healing process and risk outside contamination of the wound, damage or an injury associated with the wound cover. Indeed, the wound cover of the present inventions may provide biological data which is/are representative of a current state (and/or change in state) of the wound, damage or an injury to the body of an animal under those circumstances where suitable inspection of a covered site associated with the wound is problematic, costly and/or time consuming—for example, where the wound, damage and/or an injury is located beneath or is covered by a “fixed” solid cast (e.g., a conventional plaster cast providing a hard covering that protects, stabilizes and/or immobilizes a limb and/or joint).

The sensor(s) may include one or more active-type sensor(s) that are powered (via a local and/or remote sources) and/or one or more passive-type sensor(s) that are not powered wherein each passive-type sensor detects, senses and/or measures to a biological condition of the wound of the wound site/region and/or responds to an output therefrom, for example, from the wound (e.g., a sensor that generate an output signal(s) without application of electrical power) such as a sensor that detects temperature (e.g., heat) or a change in temperature (e.g., a thermocouple, thermistor or temperature sensitive strip). Moreover, in one embodiment, the sensor(s) may be disposed in one layer of the plurality of layers of the integrated and/or a unitary structure of the wound cover. In this embodiment, the wound cover includes one layer having one or more sensors. In another embodiment, one or more sensor(s) may be disposed in each of a plurality of layers of the wound cover and, as such, in this embodiment the wound cover includes sensors in a plurality of layers, each layer including one or more sensors.

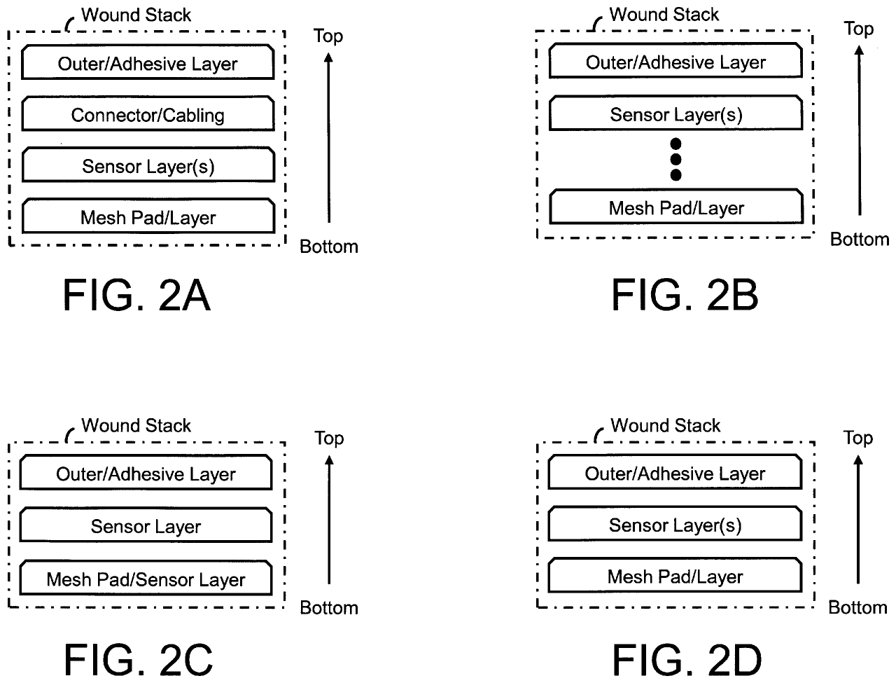

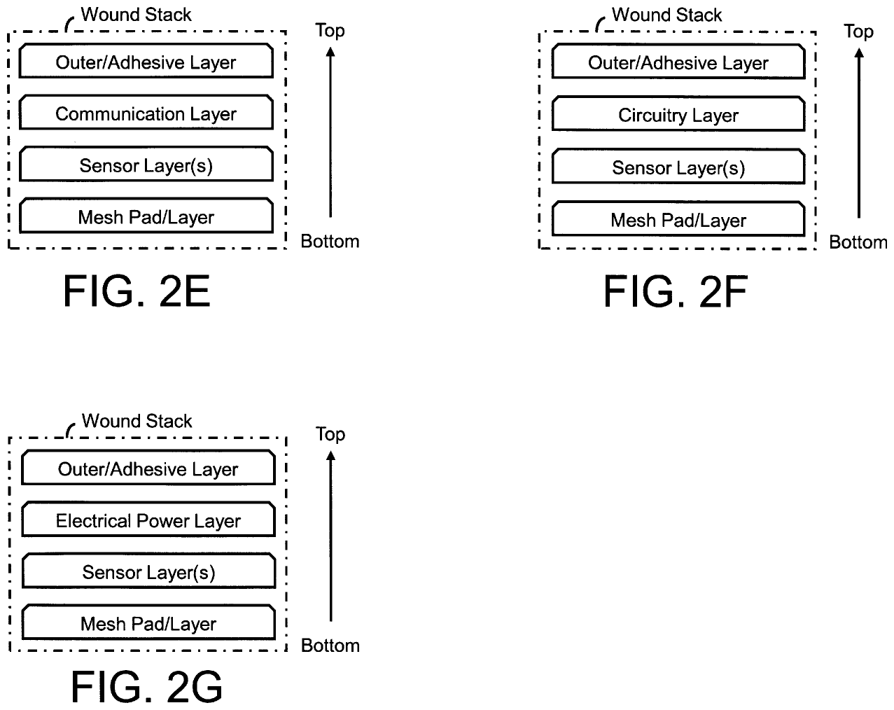

With reference to FIGS. 1A, 2B-2G and 3A, the wound stack of the wound cover includes a plurality of layers that are integrated and/or configured into a unitary structure wherein at least one of the layers includes one or more sensors capable of measuring, sensing and/or detecting physiological or biological data representative of a current state (and/or change in state) of the wound and/or wound site of the body of an animal. Notably, one or more (or all) of the layers may include a plurality of components and/or perform a plurality of functions/operations/purposes—for example, the initial layer that is intended to contact and/or directly cover the wound of the wound site (e.g., mesh pad/layer) may, in addition, include one or more sensors to detect, sense and/to measure biological data representative of a current state (and/or change in state) of the wound or wound site. (See, FIG. 2C). In that case, the mesh pad/layer is also a sensor layer. In addition, the sensor layer may also include features/components of a connector/cabling layer, a communication layer and/or a circuitry layer. Again, in that case, the sensor layer is also connector/cabling layer, a communication layer and/or a circuitry layer, as the case may be. Thus, each layer of the wound stack of the sound cover is not limited (unless expressly stated otherwise) to a single function, operation, purpose, and/or component type. (e.g., connector/cabling, sensor, communication, circuitry (e.g., processing).

Notably, with reference to FIG. 2E, the communication layer of the wound cover stack may include circuitry and/or structures to communicate data measured, detected and/or acquired by the sensors of the sensor layer to circuitry external to the wound cover. The communication layer may be configured to implement wired and/or wireless communication techniques and may include conductors, connectors, cabling, and circuitry to implement such techniques.

With reference to FIG. 2F, the circuitry layer of the wound cover stack may include circuitry layer that is configured to process (e.g., filter), evaluates and/or analyzes the physiological data, which are representative of a current state (or change in state) of the wound, output from the sensors of the sensor layer. The circuitry layer may include one or more processors, one or more state machines, one or more processors implementing software, one or more gate arrays, programmable gate arrays and/or field programmable gate arrays, or a combination of one or more circuits.

With reference to FIG. 2G, in one embodiment, the wound cover includes an electrical power layer having one or more electrical power sources resident therein/thereon (e.g., a battery) which is configured to power the circuitry of the wound cover (if any) and/or one or more sensors to, among other things, intermittently, periodically and/or continuously sense or detect biological data. In addition thereto, or in lieu thereof, in another embodiment, the wound cover is configured to receive electrical power from an external source via one or more electrical conductors or cables, which may after be distributed to the sensor(s) and/or other circuitry of the wound cover (e.g., communication component(s)). (See, e.g., FIG. 2A).

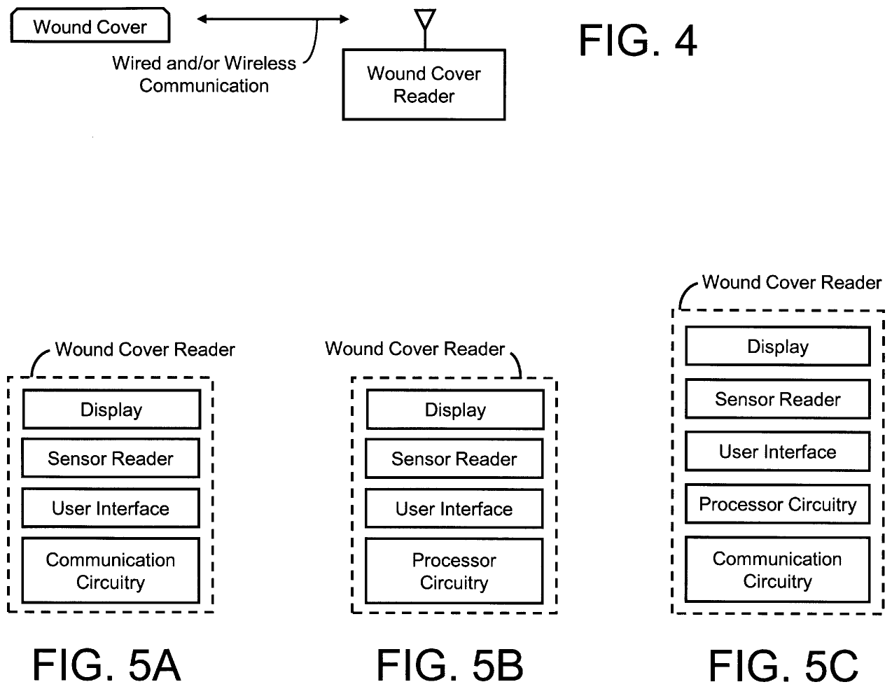

With reference to FIG. 4, in one embodiment, the present invention include a wound cover reader to optically, inductively and/or electronically detect and/or read the physiological data from the sensor(s) of the wound cover. Here, a wound cover reader (for example, a handheld device) may acquire, detect and/or read the biological data from the sensor(s), via, for example, positioning the wound cover reader in operable proximity (optically, inductively and/or electronically—e.g., 3-12 inches) to the wound cover (e.g., the sensor(s) and/or communication circuitry connected to the sensor(s)). In that way, the wound cover reader may obtain or acquire the physiological data, measured and/or detected by the sensor(s), which is/are representative of a current state (and/or change in state) of the wound and/or wound site.

In one embodiment, the one or more of the sensors may be passive-type sensor wherein the wound cover reader, via NFC techniques/circuitry and when in operable proximity, may energize, interrogate, enable, and/or access one or more sensors which, in response, may measure, collect and/or detect associated data. In addition, the sensors may transmit and/or output such physiological data (via a transmitter that is powered, for example, inductively and/or electrically via NFC techniques/circuitry) to the wound cover. In this way, the wound cover reader, when in operatable proximity to the wound cover (e.g., the sensor(s) of the sensor layer(s), may wirelessly acquire the physiological data in and/or directly from the sensor. As such, the wound cover reader may read (e.g., optically, inductively and/or electronically) the data directly from the sensor(s) and/or via communication circuitry of the wound cover—for example, (i) optically read the temperature detected by a temperature sensitive strip (e.g., the color of the strip) and/or (ii) inductively and/or electronically read the temperature detected by a thermocouple and/or thermistor (e.g., the voltage, current and/or resistance output of a thermocouple and/or thermistor, which is in operable proximity of the wound cover, via NFC techniques/circuitry).

Notably, in addition thereto, or in lieu thereof, the wound cover reader may, in one embodiment, provide one or more sensors of the sensor layer of the wound cover power/energy, via NFC techniques/circuitry, to measure, collect and/or detect associated physiological. In this embodiment, NFC techniques/circuitry implemented in the wound cover reader, when in an operable proximity of the sensor(s), provides and/or supplies power/energy to such sensor(s) (e.g., inductively and/or electronically) to sense, measure and/or detect physiological data corresponding to the sensor, and thereafter transmit and/or output such data to the wound cover reader via NFC techniques/circuitry. Thus, in this embodiment, the wound cover reader, operable proximity of the sensor(s) of the wound cover, provides or supplies the physiological sensor(s) power/energy to both sense/detect data and transmit/output that data. That is, in this embodiment, the NFC techniques/circuitry facilitates and/or implement wireless transmission of (a) electrical energy and (b) data and/or control. The NFC techniques/circuitry includes techniques that and/or circuitry configured to: (i) energize, interrogate, enable, and/or access one or more sensors which, in response, may measure, collect and/or detect associated data, and/or (ii) transmit and/or output such data (via a transmitter in the wound cover, e.g., a transmitter of or connected to one or more sensors in the wound cover) to a receiver in the wound cover reader. As such, circuitry of the NFC techniques/circuitry are disposed in both the wound cover reader as well as the wound cover.

With reference to, for example, FIGS. 1E, 1G, 2B-2D, and 3A, the exemplary wound cover implementing such a configuration may include an initial layer of porous mesh (e.g., sterile porous mesh) in contact with the wound, a second layer (e.g., disposed over and directly contacting the initial layer) having one or more sensor(s)—for example, one or more thermocouples, thermistors and/or one or more temperature sensitive strips, and an outer layer (in this exemplary embodiment, a fourth layer which is disposed over and directly contacting the third layer) having an outer adhesive edge or border to adhere the integrated, associated, and/or unitary structure of the wound cover to the skin of the animal beyond the wound site. In this embodiment, a communication component layer (e.g., disposed over and directly contacting the sensor layer) including electrical, optical and/or inductive communication component(s) may be omitted wherein the wound cover reader may be employed to facilitate acquisition of the sensor-derived biological data and, in one embodiment, may further be employed to transmit such data to circuitry external to the wound cover (e.g., local and/or remote circuitry (e.g., a signal acquisition and processing module)). Notably, again additional layers may be included between the initial/first, second, third/outer layers of this exemplary wound cover.

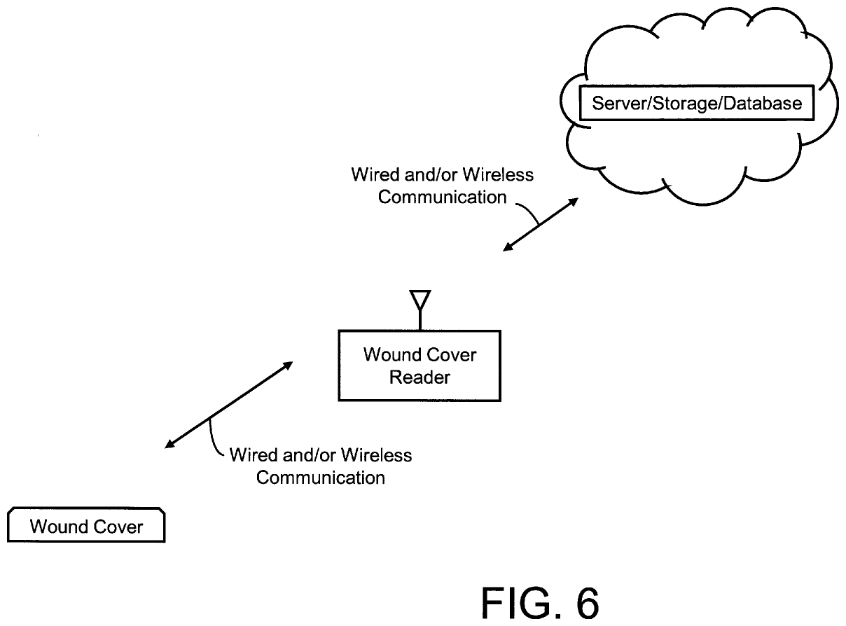

With reference to FIG. 5A-5C, in one embodiment, the wound cover reader (e.g., the handheld device) may display state/status and/or change in state/status of wound, damage or an injury to the body of an animal. In one embodiment, biological data representative of a current state (or change in state) of the wound and/or wound site thereafter be provided (for example, in real-time, e.g., via the Internet or an intranet) to a medical professional (whether local or remote) for evaluation or consideration. (See, FIG. 6). Notably, in addition to input from a medical professional, or in lieu thereof, the wound cover and system of the present inventions may employ artificial intelligence (AI) methods/processes for data analysis, trending and/or evaluation. This would be beneficial both to the patient and healthcare economic system as a whole by reducing the need for additional medical services and by returning the patient to productivity sooner. Indeed, in one aspect, the wound cover facilitates monitoring the wound and/or wound site for infection by “sensing” changes in physiologic condition of the user, for example, in real-time, continuously, intermittently and/or periodically. The monitoring operations of the wound cover, and system including the wound cover, may be aided by AI and machine learning methods/processes.

With reference to FIGS. 1A-1G, as noted above, the wound cover, according to one or more embodiments, implements a stacked component design including a wound cover stack, having both wired and wireless embodiments, including one or more, or all, of the following:
a. Layer One (having an exposed, bottom surface that is to be placed on, over and/or in contact with the wound and/or wound site (generally illustrated as a dotted box in FIG. 1B): an absorbent pad made from sterilized mesh with one or more of the following: (i) N99 grade filtration while breathable to permit gas exchange, and/or (ii) impregnation by one of more antimicrobials, including but not limited to silver, anionic/cationic biocides, or other conventional wound dressing impregnates; (iii) charcoal to absorb wound odor; and/or (iv) silicon based adhesive to allow for less peri-wound disruption upon removal. Notably, the aforementioned may be employed singly or in combination—including any combination or permutation.
b. Layer Two (disposed on and/or above Layer One): in one embodiment, a scalable sensor array with one or more (or all) of the following sensors: (i) thermistor(s) and/or thermocouples to detect a temperature of the wound and/or wound site; (ii) color sensor(s) to detect a redness of the wound and/or wound site; (iii) chemical sensor(s) to measure and/or detect local fluid abnormalities that suggest infection or tissue breakdown including, for example, pH, leukocyte esterase, urea and/or hemoglobin; (iv) bioimpedance sensors to measure and/or detect, for example, state of fluid/water in the tissue(s) of the wound and/or wound site and/or glucose metabolism of drainage of the wound and/or wound site; and/or (v) ANS tone of the wound and/or wound site. Importantly, the aforementioned sensors may be employed in the layer singly or in any combination—including any and all combinations or permutations.
c. Layer Three (disposed on and/or above Layer Two):
   wired embodiment: connectors and cabling to facilitate transmission of the sensor-derived physiological data to circuitry external to the wound cover (e.g., local and/or remote circuitry (e.g., signal acquisition and processing circuitry)); the connectors and cabling are directly connected to the sensors of the sensor layer and/or the communication component(s) of the third layer; the connectors and cabling may be affixed to or provided through the outer/adhesive layer to facilitate transmission of the sensor-derived physiological data to circuitry external to the wound cover. Notably, the aforementioned may be employed singly or in combination—including any combination or permutation.
   wireless embodiment: electrical power (e.g., a battery), signal acquisition circuitry, amplification circuitry, motion artifact reduction circuitry, processing circuitry (e.g., pre-processing (e.g., 5G artifact removal) circuitry, signal pre-processing and/or digitization circuitry), communication circuitry (e.g., Low Energy Blue Tooth transmission module), and/or low power LED indicator light. The aforementioned may be employed singly or in combination—including any combination or permutation.
d. Layer Four: (an outer layer which is disposed over Layers One, Two, Three and directly contacting Layer Three) may be an adhesive material that is configured to contain and/or maintain the wound cover stack in an integrated and/or a unitary structure; the outer layer may include an adhesive edge or border, which extends beyond the other layers of the wound cover stack, to adhere and/or temporarily affix the integrated, unitary structure of the wound cover to the wound and/or wound site and, in these illustrative embodiments, the skin of the animal in the immediate vicinity of the wound.

With reference to FIGS. 1C-1H, in certain embodiments, the wound cover is coupled to a processor module (wired or wireless). In one embodiment the processor module includes signal acquisition circuitry, amplification circuitry, motion artifact reduction circuitry, pre-processing (e.g., 5G artifact removal) circuitry, a display to visually output the status of the data representative of the state of the wound and/or wound site, signal pre-processing and digitization circuitry, communication circuitry (e.g., Low Energy Blue Tooth transmission module), and data storage and processing with controlled access and one-way communication from source (via, for example, OTA and the “cloud”).

There are many inventions described and illustrated herein. While certain embodiments, features, attributes and advantages of the inventions have been described and illustrated, it should be understood that many others, as well as different and/or similar embodiments, features, attributes and advantages of the present inventions, are apparent from the description and illustrations. As such, the above embodiments of the inventions are merely exemplary. They are not intended to be exhaustive or to limit the inventions to the precise forms, techniques, materials and/or configurations disclosed. Many modifications and variations are possible in light of this disclosure. It is to be understood that other embodiments may be utilized and operational changes may be made without departing from the scope of the present inventions. As such, the scope of the inventions is not limited solely to the description above because the description of the above embodiments has been presented for the purposes of illustration and description.

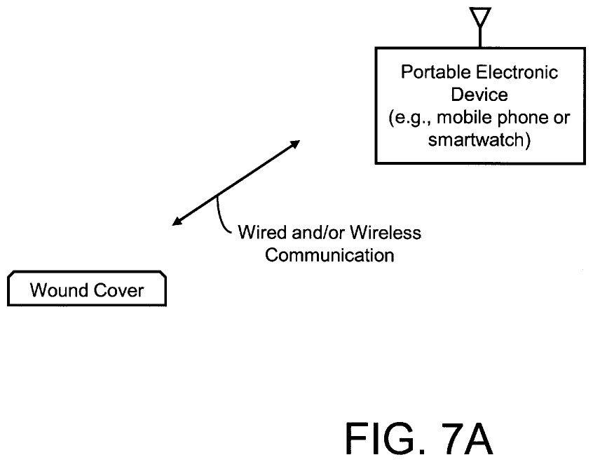

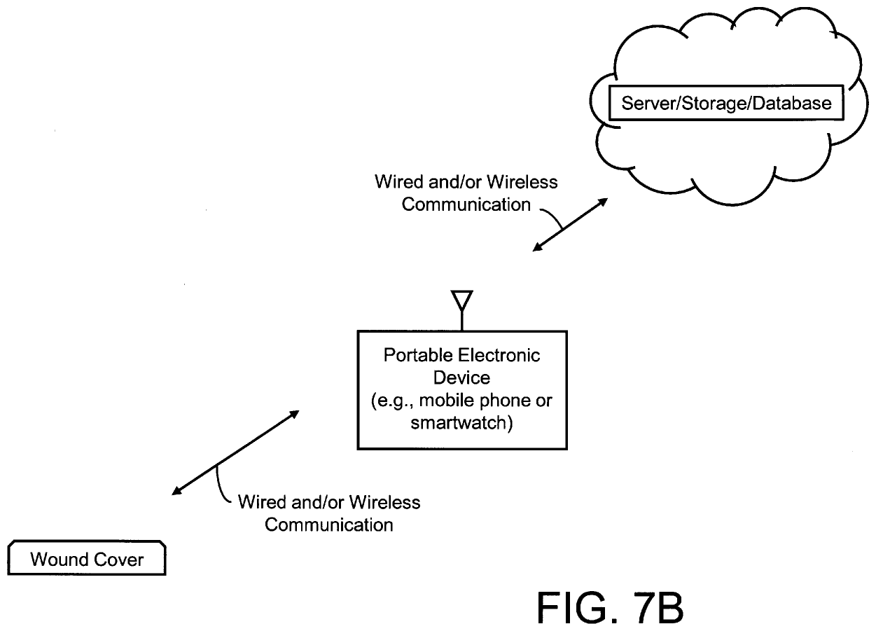

For example, with reference to FIGS. 7A and 7B, the physiological sensors of the sensor layer(s) of the wound cover, in addition to the processor module or the wound cover reader, or in lieu thereof, may communicate with a portable electronic device (e.g., personal device such as a smartwatch and/or mobile phone). The sensor data, and/or processed forms thereof, may be transmitted from the wound cover to a portable electronic device for further processing and analysis and/or display, and in one embodiment, to remote circuitry (e.g., in the cloud—see FIG. 7B) for analysis and action (e.g., implement preventive measures, corrective intervention and/or determination of appropriate treatment) and/or a smartphone, smart watch, and/or other similar technology configured for health monitoring, data collection and/or implementation of preventive measures, corrective intervention and/or determination of appropriate treatment. Notably, for the avoidance of doubt, all of the embodiments of the wound cover implemented in a system, whether employing a wired or wireless configuration, are applicable in connection with a portable/wearable electronic device (e.g., personal device such as a smartwatch and/or mobile phone)—and are intended to fall within the scope of the present inventions.

Further, although the wound cover has been described herein as being attached directly to the body (e.g., the skin) of the user via an adhesive layer having an adhesive edge or border, which extends beyond the other layers of the wound cover stack, other configurations may be employed and/or are suitable to, in operation, adhere and/or temporarily affix the integrated, unitary structure of the wound cover to the wound and/or the wound site of the user—e.g., medical tape, bandage wrap, cast and/or lightweight harness, or a combination thereof. Any technique, configuration, material and/or mechanism to temporarily affix the integrated, unitary structure of the wound cover to the wound and/or the wound site of the user, whether now known or later developed, is intended to fall within the scope of the present inventions.

In addition, although the first layer of the wound cover is often described as a mesh pad or layer (e.g., sterile porous mesh pad), the first layer of the wound cover, which includes an exposed, bottom surface that is to be placed on, over and/or in contact with the wound and/or wound site, may be an absorbent pad made from sterilized mesh, as described, and/or a layer including one or more of the following (i) N99 grade filtration while breathable to permit gas exchange, and/or (ii) impregnation by one of more antimicrobials, including but not limited to silver, anionic/cationic biocides, or other conventional wound dressing impregnates; (iii) charcoal to absorb wound odor; and/or (iv) silicon based adhesive to allow for less peri-wound disruption upon removal. Any material and/or layer that protects the wound and/or wound site of the user, whether now known or later developed, are intended to fall within the scope of the present inventions.

The communication layer of the wound cover may include circuitry and/or structures to communicate data measured, detected and/or acquired by the sensors of the sensor layer to circuitry external to the wound cover. The communication layer may be configured to implement wired and/or wireless communication techniques and may include conductors, connectors, cabling, and circuitry to implement such techniques.

In accordance with various embodiments, the present disclosure contemplates the addition of other sensor types into a sensor layer of the wound cover. Those having ordinary skill in the art will appreciate, in light of this disclosure, how various other sensors could be selected and used in conjunction with the sensors described herein to monitor for specific medical conditions of the wound and/or wound site. Importantly any sensor(s), and/or physiologic status or changes in the state of the wound and/or wound site, whether now known or later developed, are intended to fall within the scope of the present inventions.

Notably, the term “circuitry”, means, among other things, a circuit (whether integrated or otherwise), a group of such circuits, one or more processors, one or more state machines, one or more processors implementing software, one or more gate arrays, programmable gate arrays and/or field programmable gate arrays, or a combination of one or more circuits (whether integrated or otherwise), one or more state machines, one or more processors, one or more processors implementing software, one or more gate arrays, programmable gate arrays and/or field programmable gate arrays.

Importantly, the present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof.

Notably, reference herein to “one embodiment” or “an embodiment” (or the like) means that a particular feature, structure, or characteristic described in connection with the embodiment may be included, employed and/or incorporated in one, some or all of the embodiments of the present inventions. The usages or appearances of the phrase “in one embodiment” or “in another embodiment” (or the like) in the specification are not referring to the same embodiment, nor are separate or alternative embodiments necessarily mutually exclusive of one or more other embodiments, nor limited to a single exclusive embodiment. The same applies to the term “implementation.” The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, certain permutations and combinations are not discussed and/or illustrated separately herein.

Further, an embodiment or implementation described herein as “exemplary” is not to be construed as ideal, preferred or advantageous, for example, over other embodiments or implementations; rather, it is intended convey or indicate the embodiment or embodiments are example embodiment(s).

Although the present inventions have been described in certain specific aspects, many additional modifications and variations would be apparent to those skilled in the art. It is therefore to be understood that the present inventions may be practiced otherwise than specifically described without departing from the scope and spirit of the present inventions. Thus, embodiments of the present inventions should be considered in all respects as illustrative/exemplary and not restrictive.

The terms “comprises,” “comprising,” “includes,” “including,” “have,” and “having” or any other variation thereof, are intended to cover a non-exclusive inclusion, such that a process, method, circuit, system, article, or apparatus that comprises a list of parts or elements does not include only those parts or elements but may include other parts or elements not expressly listed or inherent to such process, method, article, or apparatus. Further, use of the terms “connect”, “connected”, “connecting” or “connection” herein should be broadly interpreted to include direct or indirect (e.g., via one or more conductors and/or intermediate devices/elements (active or passive) and/or via inductive or capacitive coupling)) unless intended otherwise (e.g., use of the terms “directly connect” or “directly connected”).

The terms “a” and “an” herein do not denote a limitation of quantity, but rather denote the presence of at least one of the referenced item. Further, the terms “first,” “second,” and the like, herein do not denote any order, quantity, or importance, but rather are used to distinguish one element/circuit/feature from another.

In the claims, the term “thermocouple” means or includes “thermocouple” and/or “thermistor”; for the avoidance of doubt, the same is true of the plural forms thereof. In addition, in the claims, the term “measure” means or includes “measure”, “detect” and/or “sense”; for the avoidance of doubt, the same is true of plural forms thereof.

As noted above, “wound” means a wound, damage or an injury to the body of an animal and “wound site” means a local region surrounding or adjacent to the wound. In addition, “NFC techniques/circuitry” means techniques and/or circuitry to implement wireless transmission of (a) electrical energy and/or (b) data and/or control, including techniques that and/or circuitry configured to: (i) energize, interrogate, enable, and/or access one or more sensors which, in response, may measure, collect and/or detect associated data, and/or (ii) transmit and/or output such data (via a transmitter in the wound cover, e.g., a transmitter in a sensor in the wound cover) to a receiver in the wound cover reader.

Again, there are many inventions described and illustrated herein. While certain embodiments, features, attributes and advantages of the inventions have been described and illustrated, it should be understood that many others, as well as different and/or similar embodiments, features, attributes and advantages of the present inventions, are apparent from the description and illustrations.

What is claimed is:

1. A wound cover to, in operation, be disposed on or over a wound of a user and temporarily affixed to a wound site, having the wound therein, of the user, the wound cover comprising:
a wound cover stack having a plurality of layers formed in an integrated, unitary structure including:
mesh pad layer, having (i) an exposed, bottom surface that is configured to be placed over and/or in contact with at least a portion of the wound site that includes the wound and (ii) a top surface that opposes the bottom surface of the 8 mesh pad layer;
a sensor layer, having a bottom surface and a top surface, wherein the bottom surface is disposed on the top surface of the mesh pad layer, the sensor layer having a plurality of physiological sensors, configured to measure or detect physiological characteristics of the user and, in response, generate physiological data which are representative thereof, including:
a thermocouple to detect a temperature of the wound site, and
a communication layer, disposed over the mesh pad layer and configured to wired and/or wirelessly output the physiological data measured or detected by the physiological sensors of the sensor layer to circuitry external to the wound cover; and
an outer/adhesive layer, having a bottom surface, a top surface and an adhesive edge which extends laterally beyond other layers the wound cover stack, to, in operation, temporarily affix the wound cover to the wound site of the user, wherein the bottom surface of the outer/adhesive layer is disposed over the mesh pad layer and the sensor layer of the wound cover stack.

2. The wound cover of claim 1 wherein:
the plurality of physiological sensors of the sensor layer further includes:
a chemical sensor to detect a pH of the wound site.

3. The wound cover of claim 1 wherein:
the plurality of physiological sensors of the sensor layer further includes:
a sensor to detect a blood flow in the wound site.

4. The wound cover of claim 1 wherein:
the plurality of physiological sensors of the sensor layer further includes:
a bioimpedance sensor to detect (i) a state of fluid in tissue of the wound site and/or (ii) glucose metabolism of drainage of the wound.

5. The wound cover of claim 1 wherein:
the plurality of physiological sensors of the sensor layer further includes:
a temperature sensitive strip, operatively coupled to the wound site, to responsively output a temperature and/or changes in temperature of the wound site.

6. The wound cover of claim 1 further including:
an electrical power layer to power one or more of the physiological sensors of the plurality of physiological sensors of the sensor layer.

7. The wound cover of claim 1 wherein:
the communication layer includes a connector or cabling configured to connect to the circuitry that is external to the wound cover and output the physiological data measured or detected by the physiological sensors of the sensor layer to the circuitry external to the wound cover.

8. A system to monitor the physiological conditions of a wound site, having the wound therein, of an animal, the system including:
wound cover to, in operation, be disposed on or over a wound of the animal and temporarily affixed to a wound site, having the wound therein, of the animal, the wound cover comprising:
a wound cover stack having a plurality of layers formed in an integrated, unitary structure including:
mesh pad layer, having (i) an exposed, bottom surface that is configured to be placed over and/or in contact with at least a portion of the wound site that includes the wound and (ii) a top surface that opposes the bottom surface of the mesh pad layer;
a sensor layer, having a bottom surface and a top surface, wherein the bottom surface is disposed on the top surface of the mesh pad layer, the sensor layer having a plurality of physiological sensors, configured to continuously measure or detect physiological characteristics of the animal and, in response, generate physiological data which are representative thereof; and
an outer/adhesive layer, having a bottom surface, a top surface and an adhesive edge which extends laterally beyond other layers the wound cover stack, to, in operation, temporarily affix the wound cover to the wound site of the animal, wherein the bottom surface of the outer/adhesive layer is disposed over the mesh pad layer and the sensor layer of the wound cover stack; and
a processor module, configured to wire or wirelessly coupled to the wound cover, to receive, from the physiological sensors, the physiological data which are representative of the physiological characteristics of the animal and output a status of the wound.

9. The system of claim 8 wherein:
the plurality of physiological sensors of the sensor layer of the wound cover includes a chemical sensor to detect a pH of the wound site.

10. The system of claim 8 wherein:
the plurality of physiological sensors of the sensor layer of the wound cover includes a thermocouple to detect a temperature of the wound site.

11. The system of claim 8 wherein:
the plurality of physiological sensors of the sensor layer of the wound cover includes a bioimpedance sensor to detect (i) a state of fluid in tissue of the wound site and/or (ii) glucose metabolism of drainage of the wound; and.

12. The system of claim 8 wherein:
the plurality of physiological sensors of the sensor layer of the wound cover includes a temperature sensitive strip, operatively coupled to the wound site, to responsively output a temperature and/or changes in temperature of the wound site.

13. The system of claim 8 wherein:
wound cover stack of the wound cover further includes an electrical power layer to power one or more of the physiological sensors of the plurality of physiological sensors of the sensor layer of the wound cover.

14. The system of claim 8 wherein:
wound cover stack of the wound cover further includes a communication layer having a connector or cabling configured to connect to the processor module and output the physiological data measured or detected by the physiological sensors of the sensor layer to the processor module.

15. The system of claim 8 wherein:
wound cover stack of the wound cover further includes a communication layer to wirelessly connect to the processor module and output the physiological data measured or detected by the physiological sensors of the sensor layer to the processor module.

16. A system to monitor the physiological conditions of a wound site, having the wound therein, of an animal, the system including:
wound cover to, in operation, be disposed on or over a wound of the animal and temporarily affixed to a wound site, having the wound therein, of the animal, the wound cover comprising:
a wound cover stack having a plurality of layers formed in an integrated, unitary structure including:
mesh pad layer, having (i) an exposed, bottom surface that is configured to be placed over and/or in contact with at least a portion of the wound site that includes the wound and (ii) a top surface that opposes the bottom surface of the mesh pad layer;
a sensor layer, having a bottom surface and a top surface, wherein the bottom surface is disposed on the top surface of the mesh pad layer, the sensor layer having a plurality of physiological sensors, configured to continuously measure or detect physiological characteristics of the animal and, in response, generate physiological data which are representative thereof; and
an outer/adhesive layer, having a bottom surface, a top surface and an adhesive edge which extends laterally beyond other layers the wound cover stack, to, in operation, temporarily affix the wound cover to the wound site of the animal, wherein the bottom surface of the outer/adhesive layer is disposed over the mesh pad layer and the sensor layer of the wound cover stack; and
a wound cover reader, configured to wirelessly couple to the wound cover, via NFC techniques/circuitry, when in operable proximity thereto, to receive, from the physiological sensors, the physiological data which are representative of the physiological characteristics of the animal.

17. The system of claim 16 wherein:
the plurality of physiological sensors of the sensor layer of the wound cover includes a chemical sensor to detect a pH of the wound site.

18. The system of claim 16 wherein:
at least one physiological sensor of the plurality of physiological sensors of the sensor layer of the wound cover is energized, via NFC techniques/circuitry, when the wound cover reader is in operable proximity to the wound cover.

19. The system of claim 17 wherein:
the at least one physiological sensor is a thermocouple to detect a temperature of the wound site.

20. The system of claim 16 wherein:
the plurality of physiological sensors of the sensor layer of the wound cover further includes:
a sensor to detect a blood flow in the wound site, and
a bioimpedance sensor to detect (i) a state of fluid in tissue of the wound site and/or (ii) glucose metabolism of drainage of the wound.
